(19) United States
(12) Patent Application Publication
(10) Pub. No.: US 2016/0185762 A1
(43) Pub. Date: Jun. 30, 2016

(54) METHOD FOR PRODUCING TRIS-AZIRIDINOMETHANE

(71) Applicant: BASF SE, Ludwigshafen (DE)

(72) Inventors: Joaquim Henrique TELES, Waldsee (DE); Frauke THRUN, Mannheim (DE); Peter RUDOLF, Ladenburg (DE)

(73) Assignee: BASF SE, Ludwigshafen (DE)

(21) Appl. No.: 14/909,785

(22) PCT Filed: Jul. 28, 2014
(86) PCT No.: PCT/EP2014/066119

§ 371 Date: Feb. 3, 2016

(30) Foreign Application Priority Data
Aug. 6, 2013 (EP) 13179402.6

(51) Int. Cl. C07D 403/14

(52) U.S. Cl. CPC C07D 403/14

(57) ABSTRACT

A process for preparing trisaziridinomethane (I)

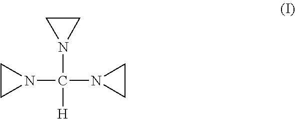

by reacting aziridine with chloroform, wherein the molar ratio of aziridine to chloroform is not more than 3:1.

The invention relates to a process for preparing trisaziridinomethane (I)

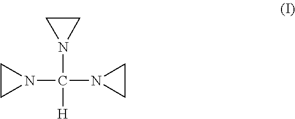

by reacting aziridine with chloroform, wherein the molar ratio of aziridine to chloroform is not more than 3:1.

Trisaziridinomethane (TAM) is a compound which, on account of its reactivity, is useful for example as a crosslinker in chemical compositions, as described in WO 03/089476.

The preparation of TAM by reacting aziridine with chloroform in the presence of a base is disclosed in W. Funke, Liebigs Ann. Chem. (1969) 725, 15. Sodium hydroxide in powder form or sodium methanolate is used as base. In this synthesis, aziridine is used in a stoichiometric excess of 6 moles of aziridine to 1 mole of chloroform. Furthermore, the total amount of aziridine is initially charged. In the reaction, a very reactive dichlorocarbene is initially formed from the chloroform and this dichlorocarbene reacts with aziridine to produce TAM. The reason why the total amount of aziridine is initially charged is so that sufficient aziridine is always available to trap the dichlorocarbene as quickly as possible. In the absence of a sufficient amount of aziridine, the reactive dichlorocarbene would stabilize itself in an alternative manner and would no longer be available for the reaction to produce TAM. The yield falls correspondingly.

In 1970, TAM was also prepared by Kostyanovskii and co-workers (Russ. Chem. Bull. (1970) 1815, translated from lzvestiya Akademii Nauk SSSR, Seriya Khimicheskaya, No 8, pp 1918-1919, August 1970) from the sodium salt of aziridine. The sodium salt of aziridine is explosive. The yield is only 22%.

Aziridine is a very reactive compound and is therefore a compound which gives rise to safety concerns. An industrial-scale synthesis therefore has to meet particular requirements. The synthesis should preclude potential risks to safety whilst simultaneously being very easy, and also very inexpensive, to carry out. TAM should be obtained by the synthesis in a very high yield and with a very high degree of selectivity.

Object of the present invention was therefore a process for preparing TAM which is suitable for performance on an industrial scale and which fulfills the above requirements to a very high degree.

It was found that this object is achieved by the process defined at the outset.

TAM is obtained from chloroform and aziridine according to the following reaction equation:

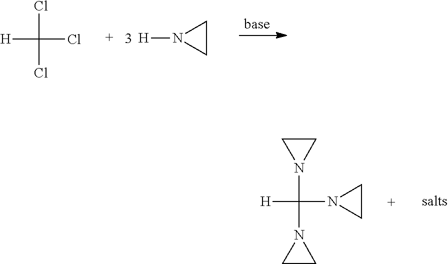

The molar ratio of aziridine to chloroform is not more than 3:1, i.e. no more aziridine is used than corresponds to the stoichiometric ratios according to the above reaction equation. In particular, aziridine can also be used in smaller amounts than the stoichiometric amounts, for example in amounts of 2.9 or 2.8 moles to 1 mole of chloroform.

Preferably, the molar ratio of aziridine to chloroform is 2:1 to 3:1; very preferably the molar ratio is 2.25:1 to 2.75:1.

In a preferred embodiment, the reaction vessel comprises not more than 50% of the total amount of the aziridine before chloroform is supplied to the reaction vessel; i.e., in the reaction, 0 to 50% of the aziridine is initially charged and the addition of chloroform to the reaction vessel is started subsequently.

In particular, the reaction vessel in which the above reaction is carried out contains 0 to 40%, very preferably 0 to 30% and most preferably 0 to 10% of the total amount of the aziridine, before the addition of chloroform is started.

Correspondingly, more than 50% of the total amount of the aziridine, in particular more than 60% of the total amount of the aziridine, very preferably more than 70% of the total amount of the aziridine and most preferably more than 90% of the total amount of the aziridine is only added once the addition of chloroform has also been started (also abbreviated to “subsequently supplied total amount of aziridine” in the following).

In a preferred embodiment, the total amount of the chloroform and the subsequently supplied total amount of aziridine (i.e. more than 50% and, in the preferred embodiments, more than 60, more than 70 or more than 90% of the total amount of the aziridine) are continuously supplied to the reaction vessel over an extended period.

The addition of the subsequently supplied total amount of aziridine can begin simultaneously with the addition of chloroform. As an alternative, the addition of only chloroform can be started initially and the addition of the subsequently supplied total amount of aziridine can be started afterwards.

Preferably, the total amount of the chloroform and the subsequently supplied total amount of the aziridine are added such that the temperature in the reaction vessel does not rise above 50° C.

In a preferred embodiment, the total amount of the chloroform and the subsequently supplied total amount of aziridine are added over an identical period of time. Very preferably, the ratio of chloroform to aziridine is held constant during the addition. This ratio then corresponds to the molar ratio of the total amount of chloroform to the subsequently supplied total amount of aziridine.

Very preferably, the total amount of the chloroform and the subsequently supplied total amount of aziridine are continuously added over an identical period of time such that the temperature in the reaction vessel does not exceed 50° C. and, in particular, does not exceed 48° C.

The above period of time can be, for example, 0.1 to 10 hours and in particular 1 to 5 hours. The period of time naturally depends on the size of the reaction vessel, the amount of the solvent used, the design of the apparatus, particularly the manner of cooling and the measures for achieving commixing (stirring means).

Preferably, the reaction is carried out in the presence of a solvent. The solvent can be initially charged to the reaction vessel or supplied during the reaction. In a preferred embodiment, the solvent is initially charged to the reaction vessel.

Preferably, a solvent is used which has a higher boiling point than aziridine. Very preferably, a solvent is used which has a boiling point at least 30° C. higher than the boiling point of aziridine. The above boiling points are the boiling points at atmospheric pressure. The solvent can also be a mixture of different solvents.

In particular, the solvent can be any aliphatic or aromatic solvent. Aliphatic or aromatic hydrocarbons or ethers, particularly aliphatic ethers such as 1,4-dioxane, tert-amyl methyl ether, cyclopentyl methyl ether, 1,2-dimethoxyethane (monoglyme) or bis(2-methoxyethyl) ether (diglyme), are preferred. Aromatic hydrocarbons, for example toluene, xylenes, ethylbenzene or mixtures thereof, are very preferred.

The solvent can be used in large amounts, for example in amounts of up to 500 parts per volume per 100 parts per volume of the total volume of the starting materials aziridine and chloroform. The larger the amount of solvent, the more favorable the heat removal to be expected during the reaction and the better the conversion and yield should be. However, it has become apparent that large amounts of solvent are not advantageous here. Therefore, for 100 parts per volume of the starting materials aziridine and chloroform, 30 to 100 parts per volume of solvent are preferred and 55 to 75 parts per volume of solvent are very preferred.

The solvent is preferably largely free of water and in a preferred embodiment comprises water at most in amounts less than 5 parts by weight, in particular in an amount less than 1 part by weight and very preferably in an amount less than 0.1 part by weight, all per 100 parts by weight of solvent. In a very preferred embodiment, the solvent comprises no water.

The reaction is preferably carried out in the presence of a base.

By way of example, alkali metal hydroxides or alkaline earth metal hydroxides can be used as the base.

In a preferred embodiment, the base is sodium hydroxide or potassium hydroxide.

Most preferably, the base, in particular sodium hydroxide or potassium hydroxide, is supplied to the reaction vessel in powder form. Preferably, the base is initially charged to the reaction vessel together with the solvent and, optionally, the abovementioned portion of aziridine.

Following the complete addition of all starting materials, the reaction is preferably continued until the desired conversion of chloroform or aziridine is achieved. In particular, the reaction is continued until more than 90%, very preferably more than 95% and most preferably more than 99% of the chloroform, in particular the total amount of the chloroform, has been consumed. In order to continue the reaction, the temperature of the reaction mixture is preferably held at 20 to 50° C., in particular at 30 to 45° C.

Following completion of the reaction, unreacted starting materials, in particular unreacted aziridine and any solvent used, for example toluene, can easily be removed by distillation from the product solution obtained.

The product solution can contain solids, in particular salts. These are, for example, unreacted base such as KOH or NaOH powder or other salts of the cation of the base used that can form under the reaction conditions, for example alkali metal chlorides or alkali metal formates.

These solids can be removed from the product solution by filtration. It has proven advantageous to carry out such a filtration after the removal of the aziridine (see above). When an aziridine-comprising product solution is filtered, aziridine can adhere to the solids that have been removed (filter cake) and complicate disposal of the filter cake.

Preferably, therefore, the product solution is filtered to remove solids after the removal of the unreacted aziridine.

The process according to the invention, for preparing TAM, is of very good suitability for performance on an industrial scale. It can be carried out easily, effectively and inexpensively and does not give rise to safety concerns.

TAM is obtained with high yield and with high selectivity using the process. This high yield of TAM is achieved using low levels of starting materials. In particular, aziridine is only used in amounts not more than stoichiometric amount.

### EXAMPLES

### Example 1

### Molar Ratio of Aziridine:Chloroform=3:1, Amount of Aziridine Initially Charged 30% by Weight

Anhydrous toluene (75 ml) and KOH powder (technical-grade, 85% by weight, 2.1 moles, 139 g) were initially charged to a 500 ml three-necked flask. One-third of the total amount of anhydrous aziridine (26.7 ml, 0.52 mole) was added thereto in one charge, with stirring. Subsequently, the chloroform (41.9 ml, 0.5 mole) and the remaining aziridine (53.5 ml, 1.04 moles) were simultaneously metered in while monitoring the temperature. The temperature did not rise above 48° C. during the addition. The ratio with which chloroform and aziridine were metered in was approximately constant over the entire period of time of the addition. After completion of the metering step, the reaction was continued at 40° C. until the chloroform had largely reacted (about 12 hours). Subsequently, the pressure was reduced and unreacted aziridine and part of the toluene were distilled off overhead. Subsequently, the solid that had precipitated out (consists predominantly of KCl, potassium formate and unreacted KOH) was removed by filtration and discarded. The conversion of the chloroform is >99% and the aziridine conversion is about 79%.

An about 25% by weight solution of TAM in toluene was obtained, comprising less than 2% by weight of free aziridine. The yield of the toluene-dissolved TAM, relative to chloroform, was 65%.

### Example 2

### Molar Ratio of Aziridine:Chloroform=2.5:1, Amount of Aziridine Initially Charged 20% by Weight

Example 2 was carried out analogously to Example 1, with the exception that the amount of aziridine initially charged was halved (13.3 ml, 0.26 mole). All other amounts remained the same. The chloroform conversion is >99% and the aziridine conversion is about 77%. Following removal of unreacted aziridine, an about 35% by weight solution of TAM in toluene was obtained, comprising less than 2% by weight of free aziridine. The yield of toluene-dissolved TAM, relative to chloroform, was 60%.

### Example 3

### Diglyme in Place of Toluene

Example 3 was carried out analogously to Example 1, with the exception that, in place of toluene, bis(2-methoxyethyl) ether (diglyme) was used as solvent. The chloroform conversion is >99% and the aziridine conversion is about 70%. Following removal of unreacted aziridine, an about 45% by weight solution of TAM in diglyme was obtained, comprising less than 2% by weight of free aziridine. The yield of toluene-dissolved TAM, relative to chloroform, was 73%. Comparative example 1 (molar ratio of aziridine:chloroform=3.6:1, amount of aziridine initially charged 14% by weight)

Comparative example 1 was carried out analogously to Example 1, with the exception that the amount of aziridine initially charged was minimized to 13.3 ml (0.26 mole) and the amount of aziridine added dropwise was increased (80.3 ml, 1.56 moles). All other amounts remained constant. The chloroform conversion is >99% and the aziridine conversion is about 70%. Following removal of unreacted aziridine, an about 40% solution of TAM in toluene is obtained, comprising less than 2% of free aziridine. The yield of toluene-dissolved TAM, relative to chloroform, was 67%.

In comparison with Example 1, it is shown that, despite the use of a large excess of aziridine, the yield of TAM remains the same. An excess of aziridine is not advantageous in the synthesis of TAM but merely increases the expense and effort associated with work-up. During the reaction of the starting materials, large temperature increases occurred, which hinders the entire performance of the reaction on an industrial scale, in particular placing great demands on the cooling system and necessitating a significant lowering of the feed rates.

### Example 4

### More Toluene

Example 4 was carried out analogously to Example 1, with the exception that the amount of toluene was increased to 100 ml. All other amounts remained the same. The chloroform conversion is >99% and the aziridine conversion is about 73%. Following removal of unreacted aziridine, an about 37% solution of TAM in toluene was obtained, comprising less than 2% of free aziridine. The yield of toluene-dissolved TAM, relative to chloroform, was 62%.

A more dilute mode of operation is not advantageous; on the contrary, with more toluene the solvent circulation streams are bloated unnecessarily and the space-time yield is lowered.

1. A process for preparing trisaziridinomethane (I) 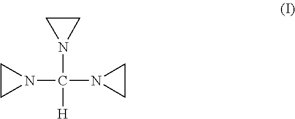
comprising reacting aziridine with chloroform, wherein a molar ratio of aziridine to chloroform is not more than 3:1.

2. The process according to claim 1, wherein the molar ratio of aziridine to chloroform is in a range from 2:1 to 3:1.

3. The process according to claim 1, wherein the reacting is performed in a reaction vessel that comprises not more than 50% of a total amount of the aziridine before chloroform is supplied to the reaction vessel.

4. The process according to claim 3, wherein the reaction vessel comprises 0 to 30% of the total amount of the aziridine before chloroform is supplied to the reaction vessel.

5. The process according to claim 1, wherein more than 50% of a total amount of the aziridine, and also a total amount of the chloroform, are continuously metered into a reaction vessel in which the reacting is performed, such that a temperature in the reaction vessel does not exceed 50° C.

6. The process according to claim 1, wherein the reacting is carried out in the presence of a solvent.

7. The process according to claim 6, wherein the solvent comprises an aromatic hydrocarbon.

8. The process according to claim 6, wherein 55 to 75 parts per volume of the solvent are used per 100 parts per volume of a total volume of the starting materials aziridine and chloroform.

9. The process according to claim 1, wherein the reacting is carried out in the presence of a base.

10. The process according to claim 9, wherein the base comprises sodium hydroxide or potassium hydroxide.

11. The process according to claim 9, wherein the base is supplied in a powder form to a reaction vessel in which the reacting is performed.

12. The process according to claim 1, further comprising removing unreacted aziridine by distillation from a product solution obtained.

13. The process according to claim 12, further comprising, following the removal of the unreacted aziridine, filtering the product solution to remove solids.
